(19) United States
(12) Patent Application Publication
(10) Pub. No.: US 2007/0197471 A1
(43) Pub. Date: Aug. 23, 2007

(54) Treatment of degenerative cartilage conditions in a mammal with Glycosidasc Inhibitors

(75) Inventor: Yoshitaka Ichikawa, San DIego, CA (US)

Correspondence Address: CATALYST LAW GROUP, APC, 9710 SCRANTON ROAD, SUITE S-170, SAN DIEGO, CA, 92121, US

(73) Assignees: Optimer Pharmaceuticals, Inc., San Diego, CA (US); The Scripps Research Institute, LaJolla, CA (US)

(21) Appl. No.: 10/586,578

(22) PCT Filed: Jan. 20, 2005
(86) PCT No.: PCT/US05/02017

§ 371 Date: Sep. 25, 2006

Related U.S. Application Data
(60) Provisional application No. 60/531,168, filed on Jan. 20, 2004.

(51) Int. Cl. A61K 31/7008; A61K 31/55; A61K 31/445; A61K 31/4745; A61K 31/4015; A61K 31/336

(52) U.S. Cl. 514/62; 514/212.01; 514/327; 514/423; 514/424; 514/475; 514/471; 514/303; 604/890.1

(57) ABSTRACT

This invention relates to treating, preventing, and lessening the severity of conditions selected from the group consisting of osteoarthritis, rheumatoid arthritis, synovitis, subchondral bone edema, and cartilage degradation with administration of glycosidase inhibitors.

This application claims the benefit of U.S. provisional application Ser. No. 60/531,168, filed on Jan. 20, 2004, which is hereby incorporated in its entirety by reference.

### FIELD OF THE INVENTION

The present invention relates to treating, preventing, and lessening the severity of conditions selected from the group consisting of osteoarthritis, rheumatoid arthritis, synovitis, subchondral bone edema, and cartilage degradation with administration of glycosidase inhibitors.

### BACKGROUND OF THE INVENTION

Synovitis, subchondral bone edema, progressive cartilage degradation and other similar conditions are separate and distinct conditions of the joints due to, among other things, physical injuries. These conditions may also be associated with osteoarthritis (OA) or rheumatoid arthritis (RA) (Ayral et. al. Rheumatology, Vol 35, 14-17; McAlindon, Best Pract Res Clin Rheumatol 1999; 13(2):329-44). For example, they may appear as secondary conditions to OA or RA or on their own due to other injuries. Osteoarthritis is the most common joint disease, currently affecting approximately 40 million Americans. This number is expected to increase to 60 million within the next 20 years. Diverse risk factors contribute to the multifactorial etiology of OA. The central pathogenetic mechanism in OA is an aberrant cartilage matrix remodeling process with loss of cartilage cells and matrix, resulting in biomechanical joint failure and inflammation.

Common in these conditions is an erosion of the cartilage. Cartilage erosion results from the over-catabolism of glycosaminoglycans (GAGs) of the proteoglycan (PG)-hyaluronate complex, which comprises the bulk of cartilage tissue. This cartilage erosion is catalyzed by glycosidases and hexosaminidases. It is known that patients with arthritis, for example, have an abnormal increase of P-N-acetylhexosaminidase activity in the synovial fluid (O. Kida, J. Japan Orthop. Assoc. 1968, 42(6), 4010; R. W. Stephen, et al., Biochim. Biophys. Acta 1975, 399(1), 101; and J. J. Steinberg, et al., Biochim. Biophys. Acta 1983, 757(1), 47). In rheumatoid arthritis, for example, the dominant glycosidases are the hexosaminidases, such as β-D-N-acetylglucosaminidase and β-D-N-acetyl-galactosaminidase. These hexosaminidases, acting either alone or in combination with other glycosidases such as β-D-glucuronidase, were shown to be directly involved in depleting GAGs from cartilage (Z. Ortutay, et al., Arthritis Rheum. 2003, 48(8), 2163).

Applicants have designed and synthesized a specific hexosaminidase inhibitor that was extremely potent, having a Ki of 24 nM against hexosaminidase and thereby preventing cytokine-induced loss of GAGs in cultured chondrocytes (J. Liu, et al., Chem. Biol. 2001, 8(7), 701; U.S. Patent Application No. 2004/0198772 A1). Further studies revealed that this inhibitor could provide a chondroprotective benefit in an osteoarthritis animal model (vide infra).

Unfortunately, the prior art does not provide for an effective means of treating, preventing, and lessening the severity of synovitis, subchondral bone edema, and cartilage degradation. Accordingly, there remains a great need for methods to treat, prevent, and lessen the severity of these conditions, which overcomes the shortcomings of the prior art.

### SUMMARY OF THE INVETION

Applicants have determined that inhibition of glycosidases in the synovial fluid has great utility as a novel chondroprotective approach in treating diseases associated with cartilage degradation. Administration of said inhibitors against these targets are useful therapeutic interventions for treating synovitis, subchondral bone edema, cartilage degradation, and other similar conditions. Therefore, inhibition of the glycosidases in synovial fluid has great utility as a novel approach to chondroprotection.

One embodiment of the present invention relates to using glycosidase inhibition to address the inflammatory and cartilage-degrading conditions of joint diseases. Said joint diseases include, but are not limited to osteoarthritis, rheumatoid arthritis, synovitis, subchondral bone edema, cartilage degradation, and other similar conditions. According to this embodiment, inhibitors of glycosidases such as hexosaminidases or glucuronidases can be used as chondroprotective agents that interfere with the breakdown of the cartilage matrix of the joint.

In another preferred embodiment of the present invention, an inhibitor of hexosaminidase, demonstrated unexpected chondroprotective effects in mammals. These chondroprotective properties give rationale to utilizing a glycosidase inhibitor(s) as a therapeutic approach for treating, for example, osteoarthritis (OA), or rheumatoid arthritis (RA).

Another preferred embodiment of the present invention contemplates the use of a single glycosidase inhibitor in combination with another glycosidase inhibitor(s) and/or another anti-inflammatory drug(s) or aminosugars for treating arthritis.

A preferred embodiment of the present invention relates to methods of treating, preventing, and lessening the severity of synovitis, subchondral bone edema, and cartilage degradation by administering to a patient a therapeutically effective amount of a glycosidase inhibitor, such as a hexosaminidase inhibitor, or glucuronidase inhibitor or a combination thereof. A therapeutically effective amount of the inhibitors can be administered to a patient by any means well known in the art, including, but not limited to orally, intravascularly, intramuscularly, topically or intra-articularly. A therapeutically effective amount of such inhibitors may also be administered intra-articularly in a matrix as a controlled release or sustained release formulation.

Another preferred embodiment of the present invention relates to a method including administering to a patient a composition containing a therapeutically effective amount of a glycosidase inhibitor (such as a hexosaminidase inhibitor(s) or a glucuronidase inhibitor(s) or a combination thereof), either alone or in combination with an existing anti-inflammatory drug or other therapeutic molecule. Methods of administering formulations of the present invention include, but are not limited to, intravascular, intra-articular, topical, oral, and intra-muscular methods.

In one embodiment of the method, a combination of glycosidase inhibitors having a specific activity or a variety of activities against hexosaminidase, glucuronidase or other endo- and exoglycosidases may also be used to achieve a chondroprotective effect in the joint.

### BRIEF DESCRIPTION OF THE DRAWINGS

FIG. 1 shows the Femur Lesion Grades determined eight (8) weeks after anterior cruciate ligament transection (ACLT) surgery for animals treated with saline or the specific hexosaminidase inhibitor ((2R,3R,4R,5R)-N-methyl-2-(acetamidomethyl)-3,4-dihydroxy-5-(hydroxymethyl) pyrrolidine), which is also known as OPT -66. Error bars represent one standard deviation.

FIG. 2 shows the Tibia Lesion Grades determined eight (8) weeks after ACLT surgery for animals treated with saline or with the specific hexosaminidase inhibitor (2R,3R,4R,SR)-N-methyl-(2-acetamidomethyl-3,4-dihydroxy-5-hydroxymethyl)-pyrrolidine, which is also know as OPT-66. Error bars represent one standard deviation.

FIG. 3 shows the Joint Swelling Grading determined eight (8) weeks after ACLT surgery for animals treated with saline or with the specific hexosaminidase inhibitor (²R,3R,4R,5R)-N-methyl-(2-acetamidomethyl-3,4-dihydroxy-5-hydroxymethyl)-pyrrolidine, which is also known as OPT-66. Error bars represent one standard deviation.

FIG. 4 shows the Synovial Fluid Grading determined eight (8) weeks after ACLT surgery for animals treated with saline or with the specific hexosaminidase inhibitor (2R,3R,4R,5R)-N-methyl-(2-acetamidomethyl-3,4-diydroxy-5-hydroxymethyl)-pyrrolidine, which is also known as OPT-66. Error bars represent one standard deviation.

FIG. 5 shows the effect of the specific hexosaminidase inhibitor (2R,3R,4R,5R)-N-methyl-(2-acetamidomethyl-3,4-dihydroxy-5-hydroxymethyl)-pyrrolidine, also known as OPT-66, has on the prevention of IL-1β-induced sGAG loss. Error bars represent one standard deviation.

### DETAILED DESCRIPTION OF THFE INVENTION

Abbreviations and Definitions

In accordance with the present invention and as used herein, the following terms and abbreviations are defined with the following meanings, unless explicitly stated otherwise. These explanations are intended to be exemplary only. They are not intended to limit the terms as they are described or referred to throughout the specification. Rather, these explanations are meant to include any additional aspects and/or examples of the terms as described and claimed herein.

The following abbreviations are used herein:

ACL=anterior cruciate ligament;

ACLT=anterior cruciate ligament transaction;

GAGs=glycosaminoglycans;

HA=hyaluronic acid;

IL1β=interleukin-1β;

IL-6=interleukin-6;

OA=osteoarthritis;

RA=rheumatoid arthritis;

sGAG=sulfated glycosaminoglycan

The term “aminosugar” refers to any synthetic or naturally occurring sugar wherein one or more carbon atoms are substituted with an amino group (—NR¹R²). Such substitution may occur without regard to orientation or configuration of any asymmetric carbons present in the sugar. Unless stated otherwise, the term “aminosugar” refers to either anomer (α or β) of a cyclic or open chain amino sugar. Aminosugars may be N-substituted with alkyl or acyl group, where one hydrogen atom of a pendant amino group is replaced by an alkyl or acyl moiety (—COR where R=alkyl or aryl). Examples of aminosugars include, but are not limited to glucosamine, N-acetyl-glucosamine, and N-acetyl-galactosamine.

The term “arthritis” refers to any particular disease characterized by joint inflammation, although the etiology of the inflammation may differ in various conditions. Relatively common arthritic diseases include rheumatoid arthritis, juvenile arthritis, ankylosing spondylitis, psoriatic arthritis, traumatic arthritis and osteoarthritis. Osteoarthritis is also referred to as “degenerative joint diseases.”

The terms “articular cartilage” or “cartilage” refer to a substance that covers ends of bones and forms the joint surfaces. Cartilage can withstand compressive forces and creates a low friction surface for a joint to glide on. Articular cartilage comprises chondrocytes and a substrate comprising proteins and glycosaminoglycan polysaccharides.

The term “anti-inflammatory agent” refers to a compound with anti-inflammatory properties, including, but not limited to, steroids (including, but not limited to, cortisone, dexamethasone, and prednisone) and non-steroidal anti-inflammatory drugs (NSAIDs) (including, but not limited to ibuprofen and naproxen).

The term “cartilage degradation” refers to degradation in the tissues comprising cartilage.

The term “chondrocyte” refers to cells found within articular cartilage. Chondrocytes produce collagen, a gelatinous protein, and proteoglycans, which are glycosaminoglycans linked to proteins (also called mucopolysaccharides).

The term “chrondroprotection” refers to a therapy designed to improve cartilage repair and enhance joint remodeling.

The term “cocktail” refers to a mixture of drugs for the treatment of a condition. Often the combined effect of the drugs in the cocktail is more potent than that of any of the drugs used individually.

The term “continuous release” is used solely to describe a drug release profile that appears to be monophasic, having a smooth-curved time profile of release. Those of skill in the art will appreciate that the release profile may actually correspond to an exponential or logarithmic time-release profile.

The term “degenerative cartilage condition” includes, but is not limited to, osteoarthritis, rheumatoid arthritis, synovitis, subchondral bone edema, and progressive cartilage degradation.

The term “glucuronic acid” refers to a derivative of D-glucose with a CO₂H group at the C-5 position that is a major component of GAGs.

The term “glucuronidase” refers to an enzyme, which releases a glucuronic acid residue from a substrate such as GAG.

The term “glucuronidase inhibitor” refers to a compound that inhibits the activity of glucuronidase. Examples of glucuronidase inhibitors include, but are not limited to, the following:

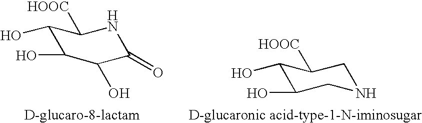

The term “glycosaminoglycan” refers to polysaccharide molecules containing repeating disaccharide units. The disaccharide units may comprise modified amino sugars: D-N-acetylgalactosamine or D-N-acetylglucosamine and an uronic acid such as D-glucuronate or L-iduronate. Among other functions, GAGs serve as a lubricating fluid in the joints. Specific GAGs of physiological significance are hyaluronic acid, dermatan sulfate, chondroitin sulfate, heparin, heparan sulfate, and keratan sulfate.

The term “glycosidase” refers to a family of enzymes involved in the processing and synthesis of complex carbohydrates which are essential for various biological recognition processes. They typically use two carboxyl groups as general acid and general base in the hydrolytic reactions. The family includes, but is not limited to, hexosaminidase and glucuronidase, which result in the catabolism of glycosaminoglycans (GAGs) of proteoglycan (PG)-hyaluronate complex.

The term “glycosidase inhibitors” refers to compounds that inhibit the activity of glycosidases. This inhibition may occur by mimicking the transition state of the enzymatic reaction of the glycosidases. Glycosidase inhibitors include, but are not limited to, iminocyclitol-based and non-iminocyclitol-based inhibitors. Target glycosidases include, but are not limited to, hexosaminidase and glucuronidase.

The term “hexosaminidase” refers to an enzyme that releases a hexosamine unit, e.g., GlcNAc and GlcN. Exemplary enzymes include exo-type beta-D-glucosoaminidase, beta-N-acetylhexosaminidase, chitosanase, chitinase, lysozyme, etc.

The term “hexosaminidase inhibitor” refers to a compound that inhibits the activity of a hexosaminidase. Examples of hexosaminidase inhibitors include, but are not limited, to the following:

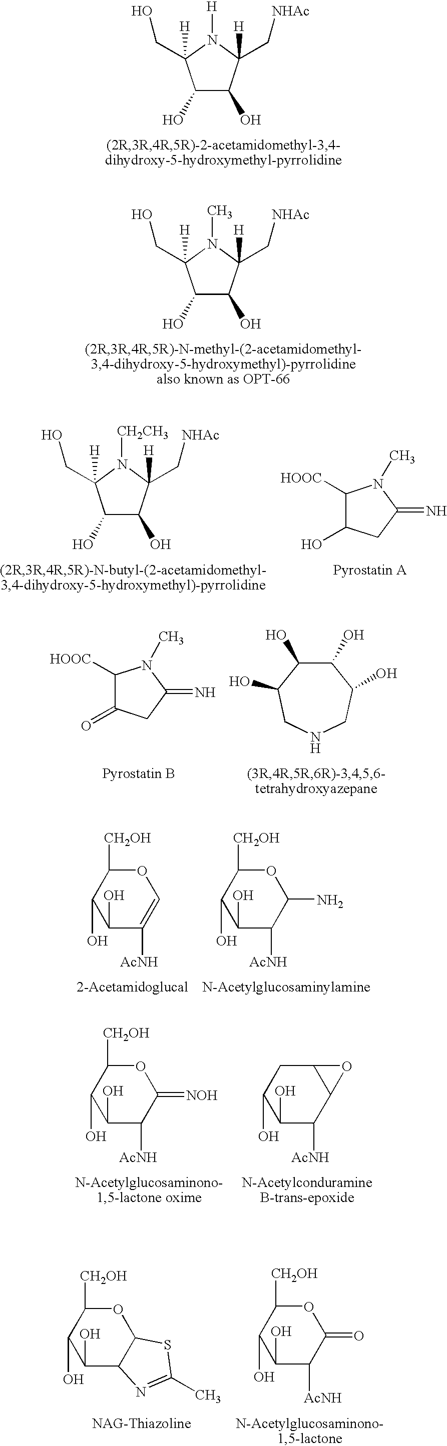

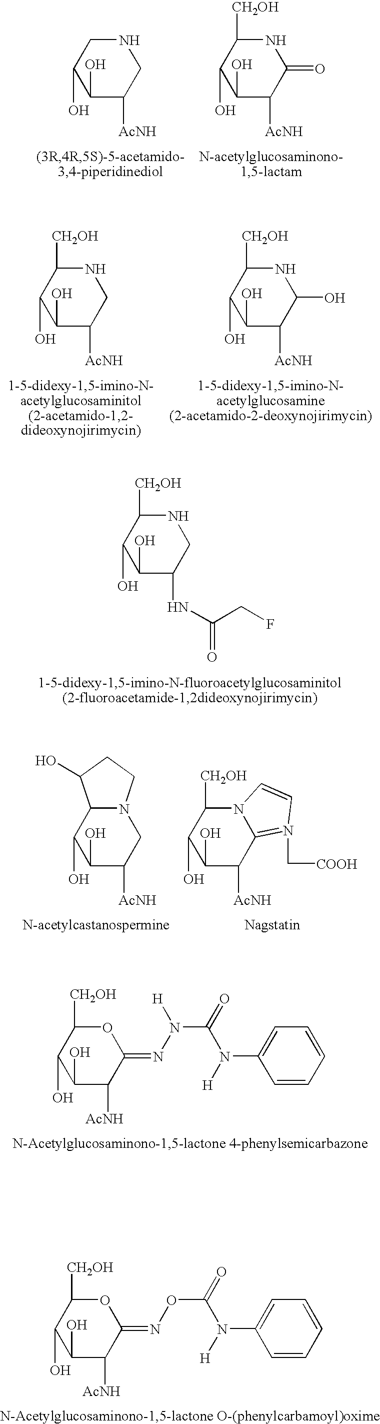

The term “hyaluronic acid” refers to a naturally occurring linear polysaccharide formed by repeating disaccharide units consisting of D-glucuronic acid β(1-3) N-acetyl-D-glucosamine linked by β(1-4) glycosidic linkages.

The term “iminocyclitol” refers to a ring structure with a nitrogen (N) as a ring member and at least one hydroxyl (OH) group attached to a ring carbon (C).

The term “iminocyclitol-based glycosidase inhibitor” refers to an iminocyclitol compound that inhibits the activity of a glycosidase enzyme. Examples of iminocyclitol-based glycosidase inhibitors include, but are not limited to the following 5-, 6-, and 7-membered ring iminocyclitols:

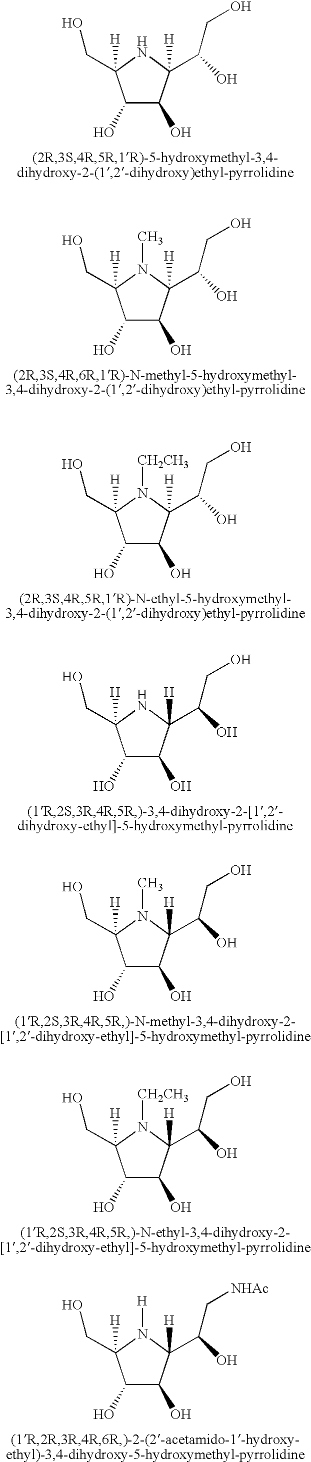

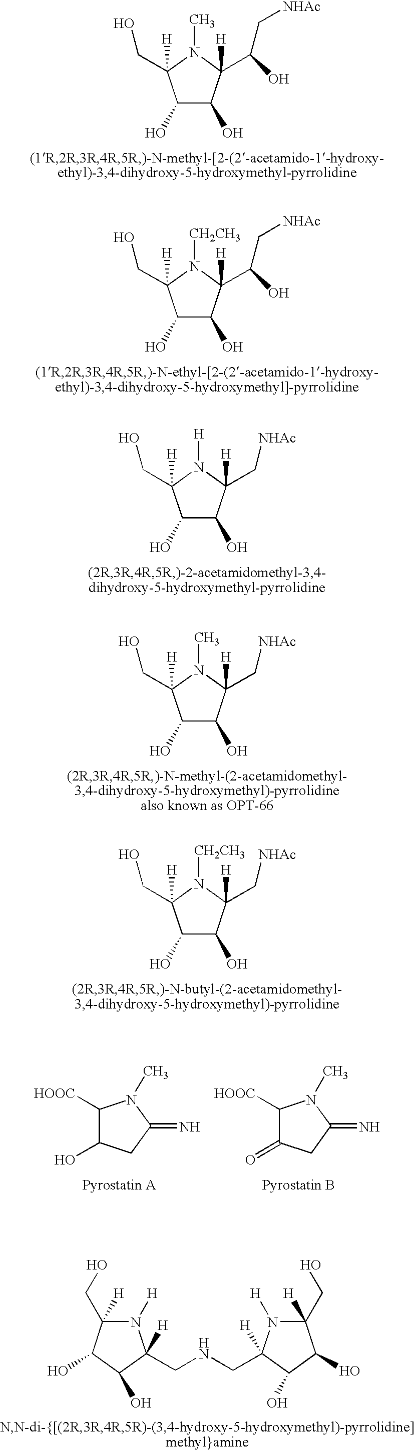

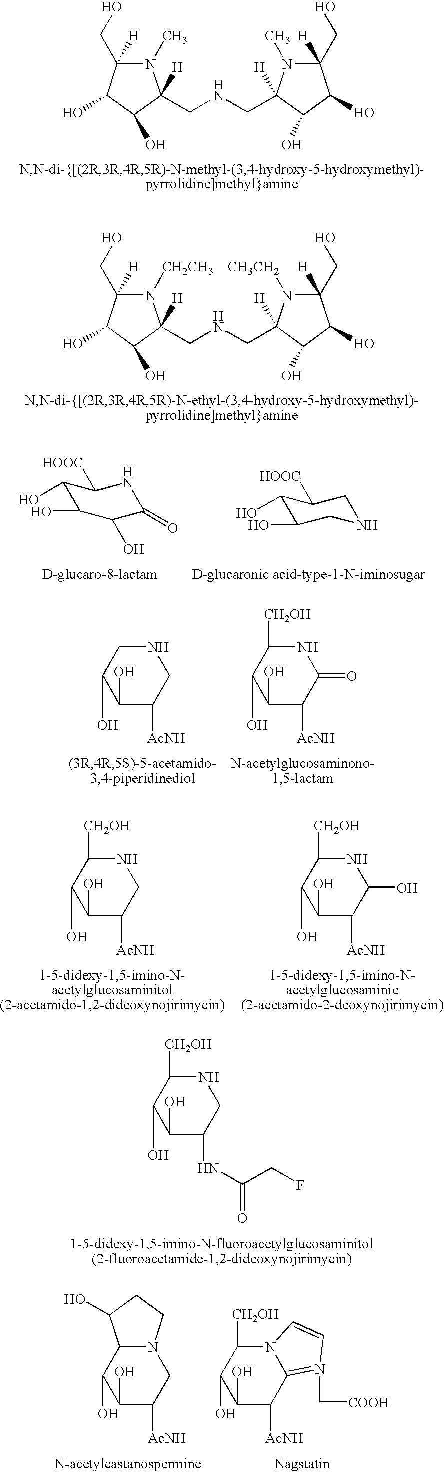

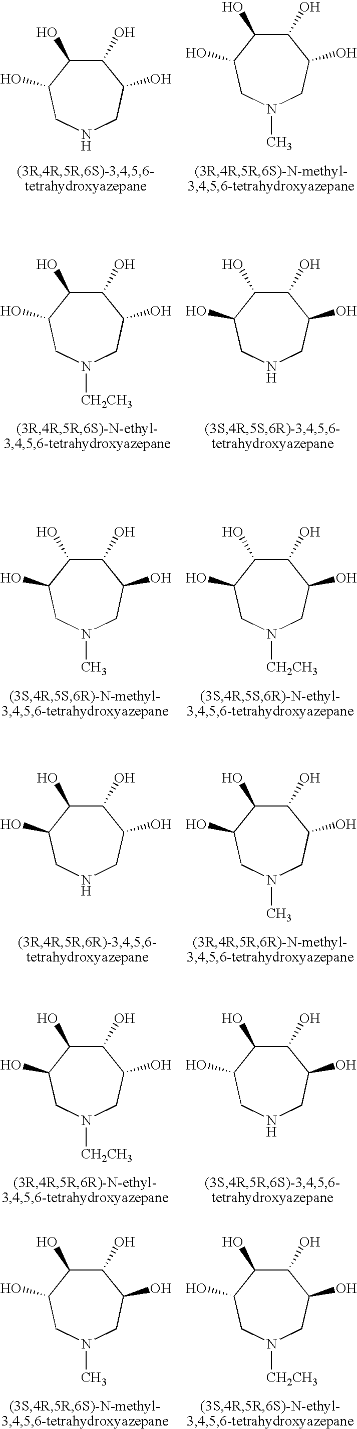

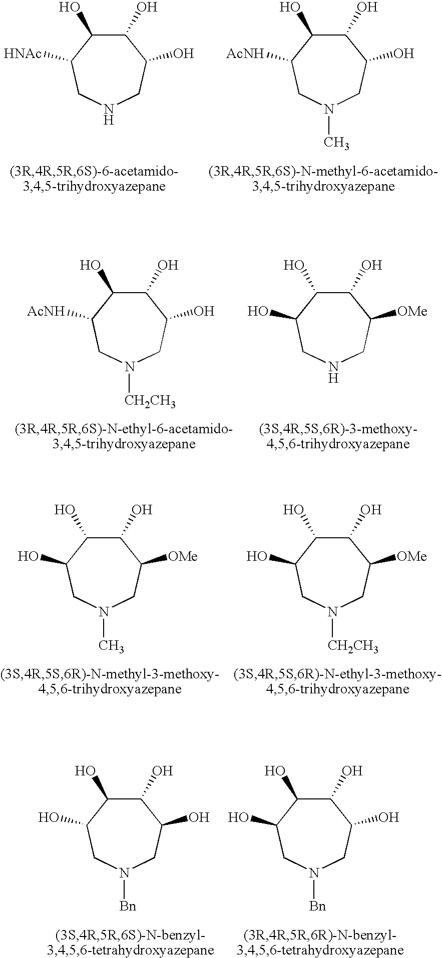

The term “IL-1β” refers to interleukin-1β, an immunomodulator that mediates a wide range of immune and inflammatory responses, including the activation of B- and T-cells.

The term “intra-articular” refers to a method of delivering a drug directly to a joint. Traditional routes of drug delivery, such as for example, oral, intravenous or intramuscular administration, depend upon vascular perfusion of the synovium to carry the drug to the joint. This is inefficient because trasynovial transfer of small molecules from the synovial capillaries to the joint space generally occurs by passive diffusion, which becomes less efficient with increasing size of the target molecule. Thus, the access of directing molecules, for example, glucosamine, to the joint space is substantially restricted. Intra-articular injection or perfusion of drugs circumvents such limitations.

The term “less severe” refers to a particular grade in cartilage degradation of a patient. Preferably, less severe grade is in the range of Grade 1 to Grade 3. More preferably, less severe grade is in the range of Grade 1 to Grade 2.

The term “non-imminocyclitol-based glycosidase inhibitor” refers to a compound that does not contain an iminocyclitol and that inhibits the activity of a glycosidase. Examples of non-iminocyclitol-based glycosidase inhibitors include, but are not limited to the following:

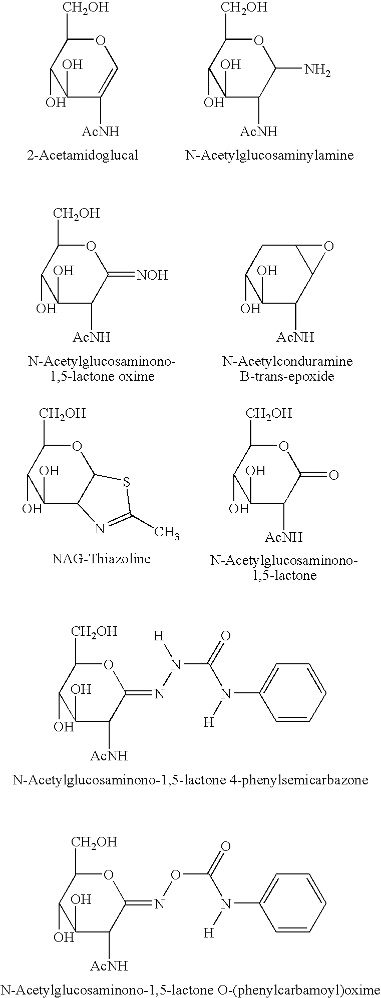

The term “OPT-66” refers to the compound (2R,3R,4R,5R)-N-methyl-(2-acetamidomethyl-3,4-dihydroxy-5-hydroxymethyl)-pyrrolidine. The structure for OPT-66 is:

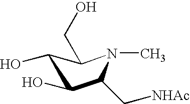

The term “proteoglycan” refers to a heavily O-glycosylated protein that gives strength to the extracellular matrix.

The term “sustained release” refers to the time period during which a drug is released for availability, or otherwise becomes available for physiological uptake. Periods of sustained release may be preceded by an induction period, during which little or no drug is released, or may be biphasic, comprising an initial time period during which some drug is released, and a second time period during which additional drug is released.

The term “synovitis” means inflammation of the joint lining (synovium). Synovitis is present in a variety of joint related conditions, including, but not limited to osteoarthritis, physical or traumatic injury, rheumatoid arthritis and other autoimmune disorders.

The term “therapeutically effective amount” refers to the amount of a biologically active substance necessary to induce a desired pharmacological effect. The amount can vary greatly according to the effectiveness of a particular active substance; the age, weight, and response of the individual; as well as the nature and severity of the individual's symptoms. Accordingly, there is no upper or lower critical limitation with respect to the amount of the active substance. A therapeutically effective amount to be employed in the present invention can readily be determined by those ordinarily skilled in the art.

Applicants have determined that inhibition of glycosidases in the synovial fluid has great utility as a novel chondroprotective approach in treating diseases associated with cartilage degradation. Administration of said inhibitors against these targets are useful therapeutic interventions for treating osteoarthritis, rheumatoid arthritis, synovitis, subchondral bone edema, cartilage degradation, and other similar conditions. Therefore, inhibition of the glycosidases in synovial fluid has great utility as a novel approach to chondroprotection.

For example, accelerated loss of proteoglycans and glycosaminoglycans is a hallmark of osteoarthritic cartilage. Increased catabolism of proteoglycans and glycosaminoglycans compromises both the functional and structural integrity of the cartilage matrix and eventually renders the tissue incapable of resisting the compressive loads applied during joint movement (Inerot, S., et al., Biochem. J., 1978, 169(1), 143). Over time, this process leads to irreversible cartilage degeneration. Loss of articular proteoglycans in established joint disease could be more significant than the collagen loss (Mankin, H. J. et al., 1970 J. Bone Joint Surg. Am., 52(3), 424). In addition to quantitative changes, affected cartilage also undergoes certain qualitative changes. Among these changes are a disproportionately increased ratio of chondroitin 4-sulfate to chondroitin 6-sulfate; a decreased ratio of keratan sulfate to chondroitin sulfate (Mankin, H. J., et al., 1971, J. Clin. Invest. 50(8: 1712); and a decreased sulfation of the terminal residues in chondroitin and dermatan sulfate chains (Plaas, A. H., et al., 1998, J. Biol. Chem. 273(20), 12642).

Degradation of the cartilage matrix is a multifactorial process involving the degradation of glycosaminoglycans by the glycosidases and involving the action of several metalloproteinases, such as collagenases, stromelysins, aggrecanases, and cysteine proteases such as cathepsins. (Winchester, B. G., 1996 Subcell. Biochem. 27, 191; Kresse, H., et al. 1987, Adv. Enzymol. Relat. Areas Mol. Biol., 60, 217).

Glycosidases are produced by chondrocytes and possess an enzymatic activity towards several glycosaminoglycans. Hexosaminidase is involved in glycosaminoglycan-degrading enzyme and is released by the chondrocytes into the extracellular compartment. It is the dominant glycosidase in synovial fluid of patients with osteoarthritis (Shikhman, A. et al., 2000 Arthritis Rheum. 43, 1307).

Hexosaminidase belongs to the group of lysosomal hydrolases. Hexosaminidase catalyzes the hydrolysis of terminal, non-reducing N-acetyl-β-D-glucosamine and N-acetyl-β-galactosamine residues in glycoproteins, G_{M2}-gangliosides, and glycosaminoglycans, including chondroitin 4-sulfate, chondroitin 6-sulfate, hyaluronic acid, keratan sulfate and dermatan sulfate (Winchester, B. G. 1996 ibid.).

One embodiment of the present invention relates to using glycosidase inhibition to address the inflammatory and cartilage-degrading conditions of joint diseases. Said joint diseases include, but are not limited to osteoarthritis, rheumatoid arthritis, synovitis, subchondral bone edema, cartilage degradation, and other similar conditions. According to this embodiment, inhibitors of glycosidases such as hexosaminidases or glucuronidases can be used as chondroprotective agents that interfere with the breakdown of the cartilage matrix of the joint.

Glycosidase inhibitors can be synthesized according to the methods of U.S. Pat. No. 6,462,193 B1; U.S. Patent Application No. 2004/0198772 A1; Igarashi et al., Tetrahedron Letters 1996, 37(16), 2707; Horsch et al., Pharmacol. Ther. 1997, 76(1-3), 187; and Ichikawa et al., JACS 2000, 120(13), 3007, all of which are hereby incorporated by reference. Examples of glycosidase inhibitors include, but are not limited to the examples listed in the above definitions section. The substituents of these examples may also be modified. An example of one such modification may be the substitution of a methyl or ethyl group in the place of a hydrogen as a substituent on a nitrogen atom.

In another preferred embodiment of the present invention, an inhibitor of hexosaminidase, demonstrated unexpected chondroprotective effects in mammals. These chondroprotective properties give rationale to utilizing a glycosidase inhibitor(s) as a therapeutic approach for treating, for example, osteoarthritis (OA), or rheumatoid arthritis (RA).

Another preferred embodiment of the present invention contemplates the use of a single glycosidase inhibitor in combination with another glycosidase inhibitor(s) and/or another anti-inflammatory drug(s) or aminosugars for treating arthritis.

A preferred embodiment of the present invention relates to methods of treating, preventing, and lessening the severity of synovitis, subchondral bone edema, and cartilage degradation by administering to a patient a therapeutically effective amount of a glycosidase inhibitor, such as a hexosaminidase inhibitor, or glucuronidase inhibitor or a combination thereof. A therapeutically effective amount of the inhibitors can be administrated to a patient by any means well known in the art, including, but not limited to orally, intravascularly, intra muscularly, topically or intra-articularly. A therapeutically effective amount of such inhibitors may also be administered intra-articularly in a matrix as a controlled release or sustained release formulation.

Another preferred embodiment of the present invention, relates to a method including administering to a patient a composition containing a therapeutically effective amount of a glycosidase inhibitor (such as a hexosaminidase inhibitor(s) or a glucuronidase inhibitor(s) or a combination thereof), either alone or in combination with an existing anti-inflammatory drug or other therapeutic molecule. Methods of administering formulations of the present invention include, but are not limited to, intravascular, intra-articular, topical, oral, and intra-muscular methods.

In one embodiment of the method, a combination of glycoside inhibitors having a specific activity or a variety of activities against hexosaminidase, glucuronidase or other endo- and exoglycosidases may also be used to achieve a chondroprotective effect in the joint.

The present invention provides examples to teach how the use of a glycosidase inhibitor (such as a hexosaminidase inhibitor) can produce a desired chondroprotective effect in a pathologic condition of cartilage such as osteoarthritis, rheumatoid arthritis, synovitis, subchondral bone edema and cartilage degradation. This approach represents a novel means to prevent cartilage matrix glycosaminoglycan degradation and a new strategy for treating osteoarthritis, rheumatoid arthritis, inflammatory joint diseases, traumatic joint diseases and related pathologic conditions.

### Pharmaceutical Formulation and Administration

A glycosidase inhibitor, as the active ingredient, can be put in pharmaceutically acceptable formulations, such as those described in Remington's Pharmaceutical Sciences, 18^{th} ed., Mack Publishing Co., Easton, Pa. (1990), incorporated by reference herein, and used for specific treatment of diseases and pathological conditions with little or no effect on healthy tissues. The preparation of a pharmacological composition comprising active ingredients dissolved or dispersed therein need not be limited based on formulation. Such compositions may be prepared as injectable liquid solutions or suspensions. However, solid forms suitable for dissolution, or resuspension, in liquid prior to use can also be prepared. The preparation can also be emulsified.

In a preferred embodiment, the composition is held within a container, which includes a label stating to the effect that the composition is approved by the FDA in the United States (or other equivalent labels in other countries) for treating a disease or condition described herein. Such a container will provide therapeutically effective amount of the active ingredient to be administered to a host.

The particular glycosidase inhibitor(s) that affects the conditions of interest can be administered to a mammal either alone or in pharmaceutical compositions where it is mixed with suitable carrier(s) or excipient(s). In treating a mammal exhibiting a condition of interest, a therapeutically effective amount of a glycosidase inhibitor is administered. The active ingredient can be mixed with excipients that are pharmaceutically acceptable and compatible with said active ingredient and in amounts suitable for use in the therapeutic methods described herein.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate administration of the compound, for example, to increase the solubility of the compound. Examples of carriers and excipients include calcium carbonate, calcium phosphate, various sugars or types of starch, cellulose derivatives, gelatin, vegetable oils, polyethylene glycols, water, saline, dextrose, glycerol, ethanol and physiologically compatible solvents.

Compositions used in the methods of the present invention can include pharmaceutically acceptable salts of the components therein. Pharmaceutically acceptable salts include acid addition salts that are formed with inorganic acids such as, for example, hydrochloric or phosphoric, sulfuric acids, etc., or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-aminoethanol, histidine, procaine and the like.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized.

For any glycosidase inhibitor used in the methods of the invention, the therapeutically effective dose can be estimated initially from cell culture assays. For example, a dose can be formulated in animal models to achieve a circulating plasma concentration range that includes the IC50 as determined in cell culture (i.e., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC.

Another preferred embodiment of the present invention relates to encapsulation or entrapment of a glycosidase inhibitor in liposomes or other entrapping agents modifies its pharmacodynamic profile when intra-articularly injected. Preferably, the glycosidase inhibitor is entrapped in a matrix. More preferably, the glycosidase inhibitor is entrapped in a matrix selected from the groups consisting of a particle, an implant, or a gel.

The exact formulation, route of administration and dosage can be chosen by the individual physician in view of the mammal's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual mammal. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990), which is incorporated herein by reference.

For injection, the agents of the invention may be formulated in aqueous solutions, preferably in physiologically compatible buffers such as Hanks's solution, Ringer's solution, or physiological saline buffer.

Use of pharmaceutically acceptable carriers to formulate the glycosidase inhibitors used in the methods herein disclosed for the practice of the invention into dosages suitable for systemic administration is within the scope of the invention. With proper choice of carrier and suitable manufacturing practice, the glycosidase inhibitors used in the methods of the present invention, in particular, those formulated as solutions, may be administered parenterally, such as by intravenous injection.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

### EXAMPLES

The following examples are provided by way of describing specific embodiments of the present invention without intending to limit the scope of the invention in any way.

### Example 1

### Effect of Continuous Infusion of a Hexosaminidase Inhibitor in an Osteoarthritis Animal Model

The model used in this study is the transection of the anterior cruciate ligament (ACL) in the rabbit knee. ACL transection (ACLT) causes joint instability and subsequent development of degradative and osteoarthritis-like changes.

Female New Zealand White rabbits, 3.0-3.5 kg, were used and were randomly allocated into groups of 8 rabbits. Group A was the saline-treated control group; Group B was treated with 30 mM of the hexosaminidase inhibitor (2R,3R,4R,5R)-N-methyl-(2-acetamidomethyl-3,4-dihydroxy-5-hydroxymethyl)-pyrrolidine, which is also known as OPT-66. All the compounds were delivered by a 2ml Alzet osmotic pump (Alzet 2ML4, Alza, USA). The delivery rate from the pump was 2.5 μl/hour. All rabbits received ACLT surgery on the right knee.

All rabbits were anesthetized by an intramuscular injection of ketamine (35 mg/kg) and acepromazine (2.5 mg/kg). Knees were shaved and disinfected with Antibex (Vetoquinol S.A.) solution. A medial parapatellar incision was made on the skin and a medial arthrotomy was performed. The patella was dislocated laterally and the knee was placed in full flexion. The ACL was visualized and transected with a #15 blade. Complete transection was confirmed by a manual anterior drawer test. The joint was irrigated with sterile saline then closed. Before the closure of knee joint, an Alzet pump prefilled with compounds (either with saline or with the hexosaminidase inhibitor (2R,3R,4R,5R)-N-methyl-(2-acetamidomethyl-3,4-dihydroxy-5-hydroxymethyl)-pyrrolidine, also known as OPT-66, was implanted subcutaneously in the lower right abdomen of rabbits. The pump was connected by polyethylene tubing (ID: 0.58 mm), which was inserted into the right knee joint with its tip resting in the synovial space. The joint capsule was closed with a running suture and the tubing connecting the pump was also fixed into the tissue. The skin was closed with interrupted sutures. The Alzet pumps were replaced with fresh units at the end of the fourth week after the operation.

The animals were sacrificed eight (8) weeks after ACLT. The gross morphological changes of both knees, including joint swelling and joint fluid, were evaluated. The occurrence, site and severity of lesions on the surface of the femurs and tibia were determined during observations under a dissecting microscope using the following criteria: Grade 1 (Intact surface), surface is normal in appearance and does not retain Indian ink; Grade 2 (Minimal fibrillation), surface retains India ink as elongated specks or light gray patches; Grade 3 (Overt fibrillation), areas which are velvety in appearance and retain India ink as intense black patches; Grade 4 (Erosion), loss of cartilage exposing the underlying bone.

The grading of the joint swelling is as following: 0 (normal); 1 (mild), inflammation and/or proliferation of the joint capsule; 2 (moderate), thickening of joint capsule and/or inflammation of the synovium; 3 (severe) abundant inflammation of the synovium, swelling of the menisci or ligaments (anterior or posterior cruciate ligaments).

The grading of the joint fluid is as following: 0 (normal); 1 (mild) fluid is greater than normal, but does not fill the knee joint; 2 (moderate) fluid fills the knee joint, but does not pour out of the capsule as it is opened; 3 (severe) fluid expands the knee joint and pours out as the capsule is opened.

Observations on the knee tissues revealed that treatment with the hexosaminidase inhibitor (2R,3R,4R,5R)-N-methyl-(2-acetamidomethyl-3,4-dihydroxy-5-hydroxymethyl)-pyrrolidine, also known as OPT-66, resulted in consistently less chondro-degenerative pathology to the knee joint, compared to saline-treated animals.

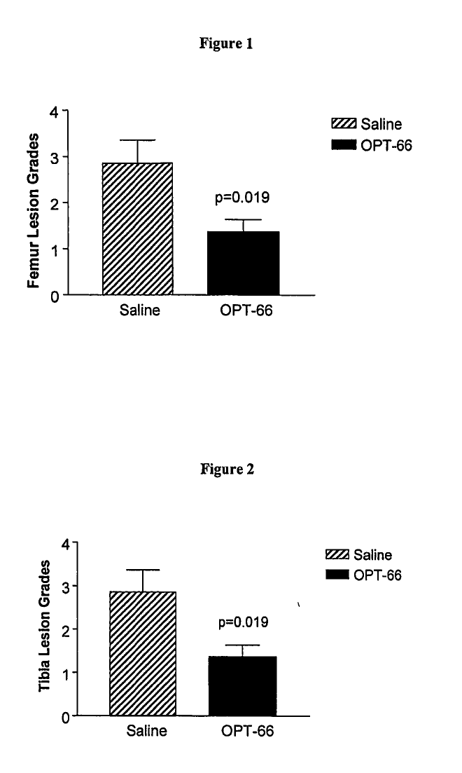

FIGS. 1 and 2 show that treatment with the hexosaminidase inhibitor (2R,3R,4R,5R)-N-methyl-(2-acetamidomethyl-3,4-dihydroxy-5-hydroxymethyl)-pyrrolidine, also known as OPT-66, leads to a statistically significant decrease in the severity of femur and tibia lesion scores, respectively, compared to saline-treated animals.

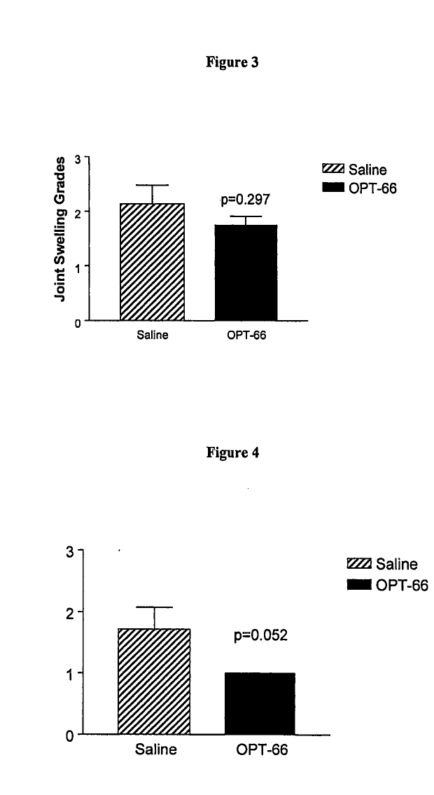

FIG. 3 shows that treatment with the hexosaminidase inhibitor (2R,3R,4R,5R)-N-methyl-(2-acetamidomethyl-3,4-dihydroxy-5-hydroxymethyl)-pyrrolidine, which is also known as OPT-66, produces a trend towards decreased joint-swelling, compared to saline-treated animals.

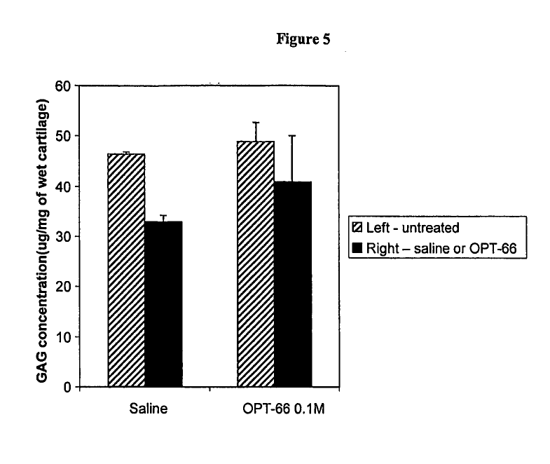

FIG. 4 shows that treatment with the hexosaminidase inhibitor (2R,3R,4R,5R)-N-methyl-(2-acetamidomethyl-3,4-dihydroxy-5-hydroxymethyl)-pyrrolidine, also known as OPT-66, results in normal levels of fluid in the knee joint. In contrast, there were increased effusions in the knees of the saline-treated animals. The scoring difference between the (2R,3R,4R,5R)-N-methyl-(2-acetamidomethyl-3,4-dihydroxy-5-hydroxymethyl)-pyrrolidine group and the saline-treated group was statistically significant.

### Example 2

### Reduction of sGAG loss by Continuous Infusion of a Hexosaminidase Inhibitor

Female New Zealand White rabbits, 3.0-3.5 kg, were used and were randomly allocated into groups of 8 rabbits. Under aseptic conditions, 2 ml Alzet osmotic pumps (delivery flow rate: 10 μl/h, (Alzet 2ML1, Alza, USA)) were filled with IL-1β (1000 U/ml; R&D Systems, USA). Separate 2 ml Alzet pumps were filled with 30 mM of the hexosaminidase inhibitor (2R,3R,4R,5R)-N-methyl-(2-acetamidomethyl-3,4dihydroxy-5-hydroxymethyl)-pyrrolidine, also known as OPT-66, or with saline. The Alzet pumps were implanted subcutaneously in the lower abdomen of rabbits, and were connected by a polyethylene tubing (ID: 0.025 in) threaded subcutaneously to the left knee joint with their tips resting in the synovial space. The untreated contralateral knees of all animals served as negative controls. IL-1β and (2R,3R,4R,5R)-N-methyl-(2-acetamidomethyl-3,4-diydroxy-5-hydroxymethyl)-pyrrolidine were infused intra-articularly for 7 days. Immediately before surgery, and for 3 consecutive days thereafter, Marbocyl, 6 mg/kg/day, is administered (i.m.) to prevent infection. Animals were harvested on day 7.

Rabbits were sacrificed by i.v. injection of pentobarbital. Both knee joints were removed and the articular cartilages of lateral tibia plateaus were harvested using a scalpel while visualized under a dissection microscopy. The cartilages were weighed and stored at −80° C. For analysis, the samples were thawed just prior to the assay. sGAGs were extracted from the sample using freshly prepared papain (1 mg papain in 2 ml of 50 mM phosphate buffer, pH 6.8 (PBS), containing 1.0 M NaCl, 5 mM, cysteine-HCl and 1 mM EDTA). 1 mg cartilage was digested with 20 microliter PBS-papain solution while stirred at 60° C. for 24 hrs. After digestion, the samples were centrifuged at 10,000 rpm for 10 min, the supernatants then were diluted 75 times with PBS-papain buffer then used in the following assay.

Blyscan dye (Biocolor, USA) was added to each tube and vortexed. The solution was mixed at 90 rpm using a mechanical shaker for 30 minutes then centrifuged at 20,000×g. The pellets were reconstituted with 450 μl of Blyscan dissociation reagent and vortexed for at least 3 min to fully dissolve the pellets. The absorbance of the sample was then observed at 656 nm using a spectrophotometer. Ultra pure water was used as blank. The absorbance of the blank was subtracted from each sample. Results are expressed as micrograms of sGAG recovered per milligram wet weight of cartilage tissue.

The recoverable sGAG content in the cartilage of the left tibial plateau of untreated knees was about 46 micrograms per milligram of wet weight of tissue (FIG. 5). Following infusion, the amount of recoverable sGAG in saline-treated knees was about 28% less than in the non-IL-1β-treated control left knee (33 μg/mg of wet weight of left tibial plateau cartilage vs. 46 μg/mg). In the (2R,3R,4R,5R)-N-methyl-(2-acetamidomethyl-3,4dihydroxy-5-hydroxymethyl)-pyrrolidine, which is also known as OPT-66, treated knees, the amount of recoverable sGAG was approximately 41 μg/mg, a 24% increase over saline-treated knees.

### OTHER EMBODIMENTS

All references discussed above are herein incorporated by reference in their entirety for all purposes. While this invention has been particularly shown and described with references to preferred embodiments thereof, it will be understood by those skilled in the art that various changes in form and details may be made therein without departing from the spirit and scope of the invention as defined by the appended claims.

1. A method of treatment of a degenerative cartilage condition comprising the step of administering a sufficient quantity of at least one glycosidase inhibitor.

2. The method of claim 1, wherein said treatment is selected from the group consisting of curing a degenerative cartilage condition, treating said degenerative cartilage condition, preventing said degenerative cartilage condition, and lessening the severity of said degenerative cartilage condition.

3. The method of claim 1, wherein said degenerative cartilage condition is selected from the group consisting of osteoarthritis, rheumatoid arthritis, synovitis, subchondral bone edema and cartilage degradation.

4. The method of claim 1, wherein said treatment of said degenerative cartilage condition is by the administration of a therapeutically effective amount of a cocktail.

5. The method of claim 4, wherein said cocktail is comprised of a therapeutically effective amount of at least one glycosidase inhibitor and a therapeutically effective amount of a therapeutic molecule additive.

6. The method of claim 5, wherein said therapeutic molecule additive is selected from the group consisting of anti-inflammatory agents and aminosugars.

7. The method of claim 4, wherein said cocktail is comprised of a therapeutically effective amount of at least one glycosidase inhibitor, a therapeutically effective amount of an anti-inflammatory agent, and a therapeutically effective amount of an aminosugar.

8. The method of claim 1, wherein said glycosidase inhibitor is selected from the group consisting of iminocyclitol-based glycosidase inhibitors and non-iminocyclitol-based glycosidase inhibitors.

9. The method of claim 8, wherein said non-iminocyclitol-based glycosidase inhibitor is selected from the group consisting of hexosaminidase inhibitors and glucuronidase inhibitors.

10. The method of claim 1, wherein said glycosidase inhibitor has a specific activity against a glycosidase.

11. The method of claim 10, wherein said glycosidase is selected from the group consisting of hexosaminidases, glucuronidases, endoglycosidases and exoglycosidases.

12. The method of claim 1, wherein said degenerative cartilage condition results in a further condition.

13. The method of claim 12, wherein said further condition is selected from the group consisting of rheumatoid arthritis, osteoarthritis, rheumatoid arthritis, inflammatory joint disease, and traumatic joint disease.

14. The method of claim 1, wherein said administration is by a route selected from the group consisting of oral administration, intra-vascular administration, intra-articular administration, intra-muscular administration, and topical administration.

15. The method of claim 1, wherein said glycosidase inhibitor is formulated in a sustained release formulation or a controlled release formulation.

16. The method of claim 1, wherein said glycosidase inhibitor is administered using a delivery device.

17. The method of claim 16, wherein said delivery device is an Alzet pump.

18. A method of treating an inflammatory condition comprising the step of administering to a subject in need thereof a quantity of at least one glycosidase inhibitor sufficient to treat the condition.

19. The method of claim 18, wherein said treatment is selected from the group consisting of curing said inflammatory condition, treating said inflammatory condition, preventing said inflammatory condition, and lessening the severity of said inflammatory condition.

20. The method of claim 18, wherein said administration is by a route selected from the group consisting of oral administration, intra-vascular administration, intra-articular administration, intramuscular administration, and topical administration.

21. The method of claim 18, wherein said glycosidase inhibitor is formulated in a sustained release formulation or a controlled release formulation.

22. The method of claim 18, wherein said glycosidase inhibitor is administered by a delivery device.

23. The method of claim 22, wherein said delivery device is an Alzet pump.

24. A pharmaceutical composition for the treatment of a degenerative cartilage condition, wherein said pharmaceutical composition is comprised of a therapeutically effective amount of at least one glycosidase inhibitor and a therapeutically effective amount of at least one therapeutic molecule additive.

25. The pharmaceutical composition of claim 24, wherein said therapeutic molecule additive is selected from the group consisting of anti-inflammatory agents and aminosugars.

26. The pharmaceutical composition of claim 25, wherein said pharmaceutical composition is comprised of a therapeutically effective amount of at least one glycosidase inhibitor, a therapeutically effective amount of an anti-inflammatory agent, and a therapeutically effective amount of an aminosugar.

27. (canceled)

28. The pharmaceutical composition of claim 24, wherein said treatment is selected from the group consisting of curing a degenerative cartilage condition, treating said degenerative cartilage condition, preventing said degenerative cartilage condition, and lessening the severity of said degenerative cartilage condition.

29. The pharmaceutical composition of claim 24, wherein said degenerative cartilage condition is selected from the group consisting of osteoarthritis, rheumatoid arthritis, synovitis, subchondral bone edema and cartilage degradation.

30. The pharmaceutical composition of claim 24, wherein said glycosidase inhibitor is selected from the group consisting of iminocyclitol-based glycosidase inhibitors and non-iminocyclitol-based glycosidase inhibitors.

31. The pharmaceutical composition of claim 24, wherein said non-iminocyclitol-based glycosidase inhibitor is selected from the group consisting of hexosaminidase inhibitors and glucuronidase inhibitors.

32. The pharmaceutical composition of claim 24, wherein said glycosidase inhibitor has a specific activity against a glycosidase.

33. The pharmaceutical composition of claim 24, wherein said glycosidase is selected from the group consisting of hexosaminidases, glucuronidases, endoglycosidases and exoglycosidases.

34. The pharmaceutical composition of claim 24, wherein said glycosidase inhibitor is formulated in a sustained release formulation or a controlled release formulation.

35. The pharmaceutical composition of claim 24, wherein said glycosidase inhibitor is administered using a delivery device.

36. The pharmaceutical composition of claim 35, wherein said delivery device is an Alzet pump.

37. A pharmaceutical composition for the treatment of an inflammatory condition comprising:
(a) at least one glycosidase inhibitor in a quantity sufficient to treat the inflammatory condition; and
(b) a pharmaceutically acceptable carrier.

38. The pharmaceutical composition of claim 37, wherein said treatment is selected from the group consisting of curing said inflammatory condition, treating said inflammatory condition, preventing said inflammatory condition, and lessening the severity of said inflammatory condition.

39. The pharmaceutical composition of claim 37, wherein said glycosidase inhibitor is formulated in a sustained release formulation or a controlled release formulation.

40. The pharmaceutical composition of claim 37, wherein said glycosidase inhibitor is administered by a delivery device.

41. The pharmaceutical composition of claim 40, wherein said delivery device is an Alzet pump.

42. The pharmaceutical composition of claim 25, wherein said pharmaceutical composition is comprised of a therapeutically effective amount of at least one glycosidase inhibitor and a therapeutically effective amount of an aminosugar.
